(19) United States
(12) Patent Application Publication
(10) Pub. No.: US 2023/0140891 A1
(43) Pub. Date: May 11, 2023

(54) MICROWAVE ABLATION ANTENNA BASED ON SPIRAL SLOT STRUCTURE

(71) Applicant: SOUTH CHINA UNIVERSITY OF TECHNOLOGY, Guangzhou (CN)

(72) Inventors: Quan XUE, Guangzhou (CN); Haidong CHEN, Guangzhou (CN); Sen LIN, Guangzhou (CN); Wenquan CHE, Guangzhou (CN)

(73) Assignee: SOUTH CHINA UNIVERSITY OF TECHNOLOGY, Guangzhou (CN)

(21) Appl. No.: 17/790,136

(22) PCT Filed: Oct. 30, 2020
(86) PCT No.: PCT/CN2020/125575

§ 371 Date: Dec. 22, 2022

(30) Foreign Application Priority Data
Dec. 31, 2019 (CN) 201911410678.6

(51) Int. Cl. A61B 18/18

(52) U.S. Cl. CPC A61B 18/1815; A61B 2018/00083; A61B 2018/1869; A61B 2018/1876

(57) ABSTRACT

A microwave ablation antenna based on a spiral slot, including a conical ablation needle head and a semi-rigid coaxial needle rod. A front end of the semi-rigid coaxial needle rod is interconnected with a tail end of the ablation needle head. An outer conductor behind a connection is provided with a plurality of optimizable spiral slots for radiation, and a plurality of annular slots are not formed or formed behind the spiral slots for impedance matching and radiation. Energy is transmitted along a semi-rigid co-axis and is efficiently radiated at the slots.

### TECHNICAL FIELD

The present invention relates to the field of microwave thermal ablation of tumors, and particularly to a microwave ablation antenna based on a spiral slot structure.

### TECHNICAL BACKGROUND

With development of minimally invasive technologies for tumors, medical technologies of microwave ablation have been gradually accepted and widely applied in the clinical medical field. Microwave ablation is one of the in-situ ablations. In-situ ablation treatment refers to a minimally invasive treatment means of focally deactivating target tissues by way of directly inputting chemical energy or non-chemical energy under guidance of imageological method CT or ultrasound as a metal needle or an electrode arrives at the target tissues by virtue of percutaneous puncture. The microwave ablation technology features large ablation range, few complications and safety, and has become a conventional treatment means for malignant tumors. Microwave is a high frequency electromagnetic wave. Transferred electromagnetic energy can be absorbed by human tissues and is then rapidly converted into a lot of heat energy.

A current microwave ablation antenna is mainly composed of monopole, dipole and coaxial slot ablation antennas and the like based on a design of a coaxial structure. (Jiang, Y., et al., A coaxial slot antenna with frequency of 433 MHz for microwave ablation therapies: design, simulation, and experimental research. Med Biol Eng Comput, 2017. 55(11): p. 2027-2036.) By slotting an outer conductor of a co-axis of a coaxial slot antenna, microwave energy is radiated in liver. However, currently a slot is long and large in spacing, so that an energy radiation part of an ablation needle is too long, and an ablation region generated by slot radiation is ellipsoidal and is small in roundness. In addition, a tip of the antenna is less in energy, which is likely to generate a tail burning effect.

### SUMMARY OF THE INVENTION

### Technical Problem

### Solution to the Problem

### Technical Solutions

The present invention overcomes the abovementioned defects and shortcomings and provides a microwave ablation antenna based on a spiral slot structure, so that a slot of an antenna is formed in a front end to reach impedance matching at a frequency 915 MHz or 2.45 GHz specified by ISM. Energy is concentrated at the tip of the antenna, and an ablation region is near-spherical.

The objective of the present invention is at least realized by one of the technical solutions as follows:

A microwave ablation antenna based on a spiral slot structure includes an ablation needle head and a semi-rigid coaxial needle rod, where a tail end of the ablation needle head is interconnected with a front end of the semi-rigid coaxial needle rod, and outermost layers of the ablation needle head and of the semi-rigid coaxial needle rod are covered with an insulating medium layer.

Further, the ablation needle head is conical, is internally provided with a metal cone which can be made of metal materials such as copper or silver, and is externally covered with the insulating medium layer.

Further, the semi-rigid coaxial needle rod has four layers, including an inner conductor, a medium layer, an outer conductor and the insulating medium layer; and the semi-rigid coaxial needle rod is internally provided with the inner conductor formed by a metallic cylinder, the outer side of the inner conductor is successively covered with the medium layer, the outer conductor and the insulating medium layer, and the outer conductor is formed by a metallic annular cylinder.

Further, the inner conductor of the semi-rigid coaxial needle rod is connected with the bottom of the cone of the ablation needle head, and the outer conductor is connected with the bottom of the cone of the ablation needle head, so that a closed short circuit is formed between the inner conductor and the outer conductor.

Further, the semi-rigid coaxial needle rod is provided with at least one spiral slot for radiation, with optimizable parameters on the outer conductor behind the connection with the ablation needle head, the number and parameters of the spiral slots are set according to a return loss and a boundary range of a temperature field, and the spiral slot is used for realizing multiple reflections, so that frequency resonance is within a specified ISM frequency.

Further, if the microwave ablation antenna based on the spiral slot structure is to work at two or more frequency points, the semi-rigid coaxial needle rod is provided with at least one annular slot with different lengths behind the spiral slot for impedance matching and radiation, and the length and number of the annular slots are set according to a boundary range of a temperature field and a return loss.

Further, the insulating medium layer and the medium layer are made from Teflon and have the characteristics of high-temperature resistance, high lubricity and no adhesion.

### BENEFICIAL EFFECTS OF THE INVENTION

### Beneficial Effects

Compared with the prior art, a microwave ablation antenna based on a spiral slot structure disclosed by the present invention has the following beneficial effects and advantages:
(1) The present invention is easy to process and low in cost based on an existing industrial technology;
(2) The present invention can realize multiple reflections in a transmission line, so that frequency resonance is easily within a specified ISM frequency;
(3) The present invention has few slots which are concentrated at the front end of the semi-rigid co-axis and is high in strength, and energy radiation is concentrated at the tip;
(4) The ablation region generated by the present invention is small in backward radiation relative to the ablation antenna with a multi-slot structure, and is closer to be spherical.

### BRIEF DESCRIPTION OF DRAWINGS

### Description of Drawings

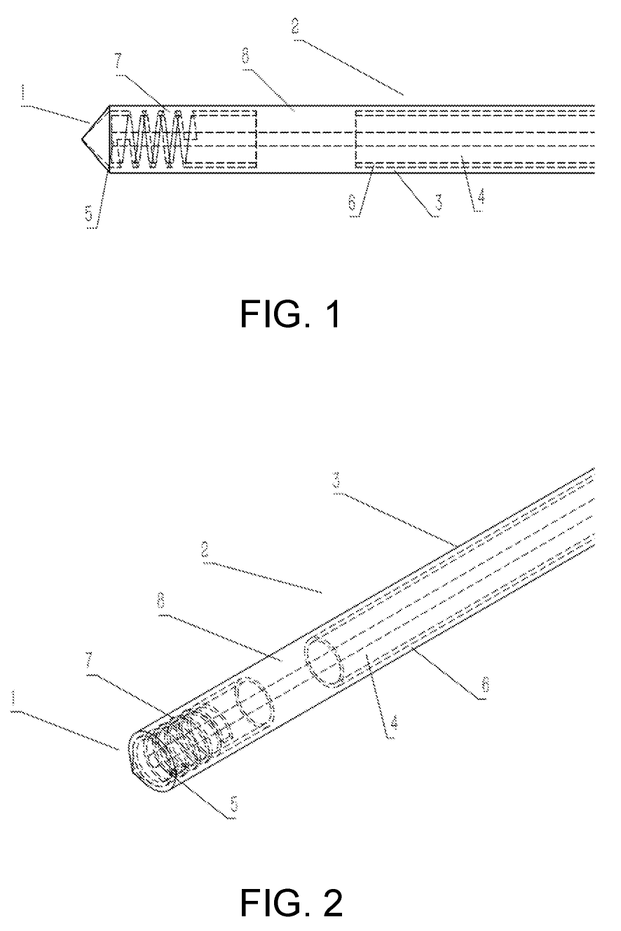

FIG. 1 is a schematic structure diagram of a microwave ablation antenna based on a spiral slot structure of the present invention.

FIG. 2 is a schematic structure diagram of an ablation needle head in an embodiment of the present invention.

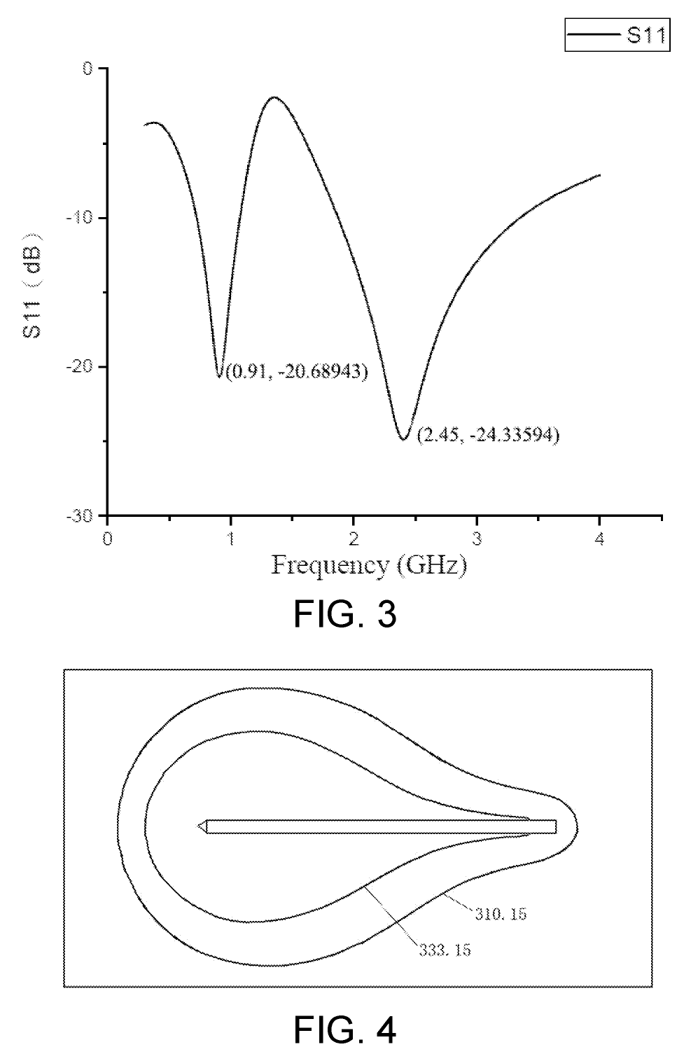

FIG. 3 is a simulation result diagram of a parameter S of a microwave ablation antenna based on a spiral slot structure in a liver in an embodiment of the present invention.

FIG. 4 is a simulation result diagram of a 2.45 GHz temperature field of a microwave ablation antenna based on a spiral slot structure in a liver in an embodiment of the present invention.

### EMBODIMENTS

### Detailed Description of Embodiments

Further description of the specific embodiments of the present invention will be made below in combination with accompanying drawings and specific embodiments. It is to be noted that the described embodiments are merely a part of embodiments of the present invention and are not all the embodiments. Based on the embodiments of the present invention, all other embodiments obtained by those of ordinary skill in the art without creative efforts shall fall within the scope of the present invention.

As shown in FIG. 1 and FIG. 2, a microwave ablation antenna based on a spiral slot structure includes an ablation needle head 1 and a semi-rigid coaxial needle rod 2, where a tail end of the ablation needle head 1 is interconnected with a front end of the semi-rigid coaxial needle rod 2, and outermost layers of the ablation needle head 1 and of the semi-rigid coaxial needle rod 2 are covered with an insulating medium layer 3.

The ablation needle head 1 is conical, is internally provided with a metal cone which can be made of metal materials such as copper or silver and is externally covered with the insulating medium layer 3.

The semi-rigid coaxial needle rod 2 has four layers, including an inner conductor 5, a medium layer 4, an outer conductor 6 and the insulating medium layer 3; and the semi-rigid coaxial needle rod 2 is internally provided with the inner conductor 5 formed by a metallic cylinder, an outer side of the inner conductor 5 is successively covered with the medium layer 4, the outer conductor 6 and the insulating medium layer 3, and the outer conductor 6 is formed by a metallic annular cylinder.

The inner conductor 5 of the semi-rigid coaxial needle rod 2 is connected with the bottom of the cone of the ablation needle head 1, and the outer conductor 6 is connected with the bottom of the cone of the ablation needle head 1, so that a closed short circuit is formed between the inner conductor 5 and the outer conductor 6.

The semi-rigid coaxial needle rod 2 is provided with at least one spiral slot 7 for radiation, with optimizable parameters on the outer conductor 6 behind the connection with the ablation needle head 1, the number and parameters of the spiral slots 7 are set according to a boundary range of a temperature field and a return loss, and the spiral slot 7 is used for realizing multiple reflections, so that frequency resonance is within a specified ISM frequency.

If the microwave ablation antenna based on the spiral slot structure is to work at two or more frequency points, the semi-rigid coaxial needle rod 2 is provided with at least one annular slot 8 with different lengths behind the spiral slot 7 for impedance matching and radiation, and the length and number of the annular slots 8 are set according to a boundary range of a temperature field and a return loss.

The insulating medium layer 3 and the medium layer 4 are made from Teflon and have the characteristics of high-temperature resistance, high lubricity and no adhesion.

### Embodiments

In the embodiment, a microwave ablation antenna of a planar structure within 2.45 GHz frequency band is designed and manufactured based on a coaxial processing technology.

FIG. 1 is a structure diagram of the embodiment of the present invention. It mainly includes an ablation needle head 1 and a semi-rigid coaxial needle rod 2. In the embodiment, the ablation needle head 1 is integrally conical, internally provided with a metal cone with a diameter of a bottom surface being 2 mm and a height being 1 mm, and externally covered with an insulating medium layer 3. The ablation needle head is made from Teflon, with enough mechanical strength and puncture force, and prevents adhesion without falling, thereby forming the cone with the diameter of the bottom surface being 2 mm and the height being 2 mm.

The semi-rigid coaxial needle rod 2 is internally provided with an inner conductor 5 of a cylindrical structure, and is externally covered with a medium layer 4, an outer conductor 6 and the insulating medium layer 3 successively. In the embodiment, a diameter of the inner conductor 5 is 0.5 mm and a length thereof is 60 mm. The medium layer 4 covering the outer side is of an annular cylinder structure and is made from Teflon, and an inner diameter of the medium layer is 0.5 mm, an outer diameter thereof is 1.7 mm and a length thereof is 60 mm. The outer conductor 6 covering the outer side of the medium layer 4 is also of an annular cylinder structure, and an inner diameter of the outer conductor is 1.7 mm, an outer diameter thereof is 2 mm and a length thereof is 60 mm. The insulating medium layer 3 covering the outer side of the outer conductor 6 is of an annular cylinder structure and is also made from Teflon, and an inner diameter of the insulating medium layer is 2 mm, an outer diameter thereof is 2.5 mm and a length thereof is 60 mm.

A front end of the semi-rigid coaxial needle rod 2 is connected with the ablation needle head 1 and is provided with a spiral slot behind the connection. In the embodiment, a distance from the spiral slot to the connection is 0.7 mm, a width of the spiral slot is 0.45 mm, a pitch is 0.3 mm, and a spiral number of turns is four. Electromagnetic waves are reflected to superpose and cancel here for many times, and an annular slot 8 is formed behind the spiral slot for impedance matching and radiation at multiple frequencies. In the embodiment, a distance from the annular slot 8 to the spiral slot 7 is 2.9 mm, a length of the annular slot 8 is 3 mm, and energy is efficiently radiated at the slot for microwave ablation.

FIG. 3 shows a simulation result of a parameter S of the microwave ablation antenna in the embodiment in liver, with resonant frequency near 915 MHz and 2.45 GHz. Within the specified ISM frequency band, they are the most common frequency bands for current microwave ablation and respectively reached -20.69 dB and -24.33 dB.

FIG. 4 is simulation of the temperature field of the microwave ablation antenna in the embodiment in a simulated liver environment, where a power was 42 W, a time was 120 s, a dielectric constant of the liver was 43, and an initial temperature of the liver was 310.15 K. A shaded area in the innermost layer in FIG. 4 is an ablation region which is higher than 333.15 K, a long diameter thereof being 66.4 mm, a short diameter thereof being 33.2 mm and a roundness being 0.5.

In conclusion, the microwave ablation antenna based on a spiral slot structure disclosed by the present invention is easy to process and low in cost based on an existing industrial technology, and realizes multiple reflections of a transmission line, so that frequency resonance is easily within a specified ISM frequency. The microwave ablation antenna based on a spiral slot structure has few slots which are concentrated at the front end of the semi-rigid co-axis and is high in strength, and energy radiation is concentrated at the tip, the ablation region generated by the present invention is small in backward radiation relative to the ablation antenna with a multi-slot structure, and is closer to be spherical.

1. A microwave ablation antenna based on a spiral slot structure, comprising an ablation needle head and a semi-rigid coaxial needle rod, wherein a tail end of the ablation needle head is interconnected with a front end of the semi-rigid coaxial needle rod, and outermost layers of the ablation needle head and of the semi-rigid coaxial needle rod are covered with an insulating medium layer.

2. The microwave ablation antenna based on a spiral slot structure according to claim 1, wherein the ablation needle head is conical, is internally provided with a metal cone and is externally covered with the insulating medium layer.

3. The microwave ablation antenna based on a spiral slot structure according to claim 1, wherein the semi-rigid coaxial needle rod has four layers, comprising an inner conductor, an outer conductor and the insulating medium layer; and the semi-rigid coaxial needle rod is internally provided with the inner conductor formed by a metallic cylinder, an outer side of the inner conductor is successively covered with the medium layer, the outer conductor and the insulating medium layer, and the outer conductor is formed by a metallic annular cylinder.

4. The microwave ablation antenna based on a spiral slot structure according to claim 3, wherein the inner conductor of the semi-rigid coaxial needle rod is connected with a bottom of the cone of the ablation needle head, and the outer conductor is connected with the bottom of the cone of the ablation needle head, so that a closed short circuit is formed between the inner conductor and the outer conductor.

5. The microwave ablation antenna based on a spiral slot structure according to claim 3, wherein the semi-rigid coaxial needle rod is provided with at least one spiral slot for radiation, with optimizable parameters such as a spiral number of turns, a spiral pitch, a width of spiral slot and a distance between the spirals on the outer conductor behind the connection with the ablation needle head, the number and parameters of the spirals are set according to a return loss and a boundary range of a temperature field, and the spiral slot is used for realizing multiple reflections, so that frequency resonance is within a specified ISM frequency.

6. The microwave ablation antenna based on a spiral slot structure according to claim 1, wherein if the microwave ablation antenna based on the spiral slot structure is to work at two or more frequency points, the semi-rigid coaxial needle rod is provided with at least one annular slot with different lengths behind the spiral slot for impedance matching and radiation, and the length and number of the annular slots are set according to a boundary range of a temperature field and a return loss.

7. The microwave ablation antenna based on a spiral slot structure according to claim 3, wherein the insulating medium layer and the medium layer are made from Teflon.
